(19) United States
(12) Patent Application Publication
(10) Pub. No.: US 2005/0106251 A1
(43) Pub. Date: May 19, 2005

(54) Zero order controlled drug delivery system

(76) Inventors: John Richard Langridge, Sint Oedeurode (NL); Wei Tian, Little Mellon (GB); Olayinka Lawal, Dartford (GB)

Correspondence Address: MCANDREWS HELD & MALLOY, LTD, 500 WEST MADISON STREET, SUITE 3400, CHICAGO, IL, 60661, US

(21) Appl. No.: 10/484,748

(22) PCT Filed: Jul. 18, 2002
(86) PCT No.: PCT/GB02/03292

(30) Foreign Application Priority Data
Jul. 19, 2001 (GB) 0117619.7

(51) Int. Cl. A61K 9/22; A61K 9/26

(52) U.S. Cl. 424/470

(57) ABSTRACT

A controlled release dosage form comprising: (i) a tablet core comprising a pharmaceutically active ingredient and one or more pharmaceutically acceptable matrix forming polymers, (ii) a substantially insoluble casing extended over the tablet core covering between 25 to 99% of the surface area of the tablet core, like for example covering only the major surfaces like in FIG. 1 or on major surface and the sidewells like in FIG. 2, the casing resulting from electrostatic deposition of a powder comprising fusible particles onto the tablet core and fusing the particles to form a thin film such that the said electrostatic coated tablet releases the active ingredient with a release profile of active ingredient for 0 to at least 50% by weight release of active ingredient defined by the equations y=k*tⁿ in which y is the fraction of active ingredient released, k is the kinetic constant, t is time, n is the release exponent and n is the range 0.70 to 1.0 i.e. an approximately zero order release profile.

The present invention relates to a controlled drug delivery system that releases an active material at a constant rate (i.e. zero order) into a biological fluid, in particular, the fluid of the gastrointestinal tract.

Tablets are often the preferred means of administering medicine to patients. A conventional immediate release tablet releases the drug active in the body rapidly reaching a maximum concentration then decaying expeditiously until the next administration. This method often leads to peaks and troughs of drug concentration in the blood and requires frequent administration of tablets. Consequently, this could lead to either exacerbated harmful side effects at high concentrations or diminished therapeutic effects at low concentrations. These effects can become acute with actives of relatively short biological half life. To counter these, controlled release dosage forms which release actives at a constant rate over a defined period of time (zero order release) have been frequently employed.

Many controlled release tablets are prepared either using a matrix system through the formation of polymer networks, or using a membrane system such as film coating. The dissolution kinetics over the time when the majority of drug is released can be represented by the following mathematical equation:
y=k*tⁿ

Where y is the fraction released

k is the kinetic constant

t is time

n is the release exponent

The release exponent n is characteristic of release mode, if n=0.5, Fickian diffusion dominates, i.e. the structural relaxation of polymer network is rapid and the rate limiting step is the self-diffusion of drug active. This is termed first order release. If n=1, the release of active is at a constant rate, i.e. zero order release. The rate-limiting step is the rate of polymer relaxation.

There are numerous factors affecting the release rate of the actives, for example the molecular weight, glass transition temperature, the swelling volume, gelation potential of the network forming polymer etc. Hence, in practice, the release rate can only be controlled to a limited extent by polymer matrix alone with the release exponent n at a value close to 0.5.

U.S. Pat. No. 4,792,448 discloses a device for the controlled release of one or more active substances into a fluid medium at a substantially constant rate (i.e. zero oder) which comprises said substance homogenously dispersed in the shape of a cylindrical tablet or bolus by means of an all-covering, essentially impermeable wall or coating except for one or more strips of removed wall or coating from the side of said device.

EP0259113 claims a device for the controlled release of one or more active substances into a fluid medium which comprises said substance homogenously disposed, with or without one or more inert diluents, and contained substantially in the shape of a truncated cone by means of an impermeable wall or coating on the base and side of said truncated cone.

U.S. Pat. No. 5,004,614 discloses controlled release devices having a core including an active agent and an outer coating which is substantially impermeable to the entrance of an environmental fluid and substantially impermeable to the release of the active agent during a dispensing period allow the controlled release of the active agent through an orifice in the outer coating. The coating thickness, the position, number and the sizes of the orifices are the key variables influencing the release profile.

U.S. Pat. No. 4,839,177 discloses a system for the controlled-rate release of active substances, consisting of: (a) a deposit core comprising the active substance and having defined geometric form; (b) a support-platform applied to said deposit core. Said deposit core contains, mixed with the active substance, a polymeric material having a high degree of swelling on contact with water or aqueous liquids, a gellable polymeric material, said polymeric materials being replaceable by a single polymeric material having both swelling and gelling properties. Said support-platform consists of a polymeric material insoluble in aqueous liquids and partially coating said deposit-core. However, these tablets have the drawback that the rigid support can result in cracking and sometimes flaking before the active substance has been completely released. This patent was superseded by U.S. Pat. No. 5,422,123, which discloses tablets with zero order controlled rate of release of the active substances, consisting of a core of defined geometrical form containing the active substance, polymer substances which swell on contact with aqueous liquids and polymer substances with gelling properties, and a support applied to said core or partly cover its surface, the support consisting of polymer substances which are slowly soluble and/or slowly gellable in aqueous liquids, plasticizing substances, and possible substances with an adjuvant function.

U.S. Pat. No. 6,033,685 provides a tablet for controlled release of an active agent consisting of (a) a matrix layer comprising an active agent embedded in non-swelling, non-gelling hydrophobic matrix; (b) a first barrier layer laminated to a single face of the matrix layer; and (c) an optional second barrier layer laminated to the opposite face of the matrix layer and oppositely disposed to the first barrier layer.

U.S. Pat. No. 6,083,533 discloses a layered tablet for controlled release of active substances in a liquid medium comprising at least one active substance containing, layered matrix with contact surfaces to the liquid medium which are at least partially provided with a cover layer delaying or preventing the active substance release, is characterised by the fact that the cover layer is at least one additional layer lying with thickness gradients on contact surfaces of the layered, prefabricated matrix.

U.S. Pat. No. 6,264,985 discloses a compression-coated tablet with an erodible core and a substantially erosion resistant shell. The shell has at least one opening and one end of the core extends as far as the opening.

WO 921445 discloses that electrostatic deposition may be used to apply a coating of controlled thickness and may be employed for a medicinal product containing a drug that is to be instantaneously released when administered or that is to be the subject of controlled or modulated release, such control of modulation being achieved from the nature of the coating and/or from the nature of core. Where the desired form of release is to be achieved by characteristics of the coating, it may be preferred to leave one portion of the product uncoated or coated with different material. In the case of a tablet having faces at opposite ends connected by a cylinder side wall, the portion that is uncoated or coated with different material may be one of the faces of the tablet, a small portion of one of the faces or a side wall of the tablets. However, there is no disclosure as to whether or how a zero order release profile can be achieved.

There is a need for an effective pharmaceutical dosage form having controlled release of an active ingredient at substantially constant rate.

In accordance with the present invention there is provided a controlled release dosage form comprising:

(i) a tablet core comprising a pharmaceutically active ingredient and one or more pharmaceutically acceptable matrix forming polymers, the tablet core releases the active ingredient with a release profile of active ingredient for 0 to at least 50% by weight release of active ingredient defined by the equations
y=k*tⁿ

in which y is the fraction of active ingredient released

k is the kinetic constant

t is time

n is the release exponent

and n is the range 0.30 to 0.65

(ii) a substantially insoluble casing extended over the tablet core covering between 25 to 99% of the surface area of the tablet core, the casing resulting from electrostatic deposition of a powder comprising fusible particles onto the tablet core and fusing the particles to form a thin film such that the said electrostatic coated tablet releases the active ingredient with a release profile of active ingredient for 0 to at least 50% by weight release of active ingredient defined by the equations
y=k*tⁿ

in which y is the fraction of active ingredient released

k is the kinetic constant

t is time

n is the release exponent

and n is the range 0.7 to 1.0 i.e. an approximately zero order release profile.

It has been surprisingly found that a pharmaceutical dosage form having controlled release of an active ingredient at a substantially constant rate, i.e. zero order release rate, can be obtained by electrostatic application of a thin film on the selected surface of a tablet. The release profile does not require the application of a thick film nor rely on the controlled thickness so long as a complete and uniform coating within the defined area is obtained. Furthermore, there are no needs for a special designed geometric shape, the mechanical removal of a portion of film coating at a defined position with a defined surface area or the presence of specific matrix forming polymers.

The invention provides a simple and effective means of producing a pharmaceutical dosage form having an approximately zero order release profile for a pharmaceutical active agent. A drug reservoir, in the form of a tablet core and having an approximately first order release profile, which may be made by conventional techniques, is provided with an insoluble casing covering 25 to 99% of the surface area of the tablet. In this manner, the area of the tablet exposed to the body fluids, e.g. gastric juices when the dosage form is administered, is reduced thereby decreasing the hydration rate of the tablet core and the drug release rate such that the resulting tablet has an approximately zero order release profile.

The electrostatic coated tablet preferably has the release profile in which n=0.7 to 1.0 over 0 to at least 50% by weight release of active ingredient, more preferably from 0 to at least 60% by weight release of active ingredient, most preferably from 0 to greater than 70% release of active ingredient. In preferred embodiments the release profile requires at least four hours, more preferably at least five hours to achieve 70% by weight release of active ingredient.

The release profile of a pharmaceutical active is determined by standard US Pharmacopoeia method using a paddle stirring element (Apparatus II), Vankel™ 7000 dissolution apparatus (Apparatus II). The assembly consists of the following: a covered vessel made of glass or other inert, transparent material; a motor; a paddle formed from a blade and a shaft. The shaft is positioned so that its axis is not more than 2 mm at any point from the vertical axis of the vessel and rotates smoothly without significant wobble. The vertical centre line of the blade passes through the axis of the shaft so that the bottom of the blade is flush with the bottom of the shaft. The distance of 25±2 mm between the paddle and the inside bottom of the vessel is maintained during the test.

The vessel is partially immersed in a suitable waterbath which maintains the temperature inside the vessel at 37±0.50° C. during the test and keeping the bath fluid in constant, smooth motion. The vessel is cylindrical, with a hemispherical bottom. Its sides are flanged at the top. A fitted cover may be used to retard evaporation. Demineralised water is added to the vessel. The dosage unit (one single tablet) is allowed to sink to the bottom of the vessel before the rotation of the blade is started. The stirring rate is set at 50 rpm. The released active ingredient with time is measured by any suitable means e.g. u.v. analysis, HPLC etc. and expressed as percentage release (w/w) of the total weight of active ingredient.

The casing extending over the tablet core results from the electrostatic deposition of a powder comprising fusible particles. This technique allows the formation of a thin, continuous casing over the tablet core. Although the release profile does not depend on the coating thickness, it is of importance that a continuous and complete coverage is applied in order to minimise pore formation. Typically this requires the deposition of several layers of powdered material (the powders have a mean diameter of 10 μm) to give a coating thickness of at least 20 μm after fusion. Generally the maximum coating thickness of the tablets is not more than 75 μm. Coating thickness in the range 20 to 50 μm is preferred. Generally the coating results in a weight gain of less than 5%, often less than 4% and frequently less than 3% by weight of the tablet core. In general, the casing will cover from 25 to 99% of the surface area of the tablet core, generally 50 to 99%, preferably 65 to 95% of the surface area of the tablet core, leaving the reminder exposed.

The shape of the tablet core is not critical since the electrostatic deposition of powder can readily be achieved over a variety of shaped bodies. The tablet core is conveniently formed by conventional tableting techniques e.g. compression of powder and/or granules, although other moulding techniques may be employed. A convenient tablet core has a circular cross-section and two major opposing surfaces which may be, for example, planar, planar with a bevelled edge, concave, convex etc. The insoluble casing may conveniently extend over one of the major surfaces and the side wall leaving the other major surface exposed.

The tablet core comprises at least one adjuvant and a pharmaceutically active-ingredient. Generally the adjuvant will comprise a binder. Suitable binders are well known and include acacia, alginic acid, carboxymethylcellulose, hydroxyethylcellulose, hydroxypropylcellulose, dextrin, ethylcellulose, gelatin, guar gum, hydrogenated vegetable oil, hydroxypropylmethylcellulose, maltodextrin, methylcellulose, polyethylene oxide, povidone, sodium alginate and hydrogenated vegetable oils.

The tablet core preferably comprises a release rate controlling additive. For example, the drug may be held within a hydrophobic polymer matrix so that it is gradually leached out of the matrix upon contact with body fluids. Alternatively, the drug may be held within a hydrophilic matrix which gradually dissolves or swells in the presence of body fluid.

Suitable release rate controlling polymers include polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, glyceryl monostearate, zein etc.

The tablet core may comprise other conventional tableting ingredients, including diluents, disintegrants, lubricants, wetting agents, glidants, surfactants, release aids, colourants, gas producers, etc. Suitable diluents include lactose, cellulose, dicalcium phosphate, sucrose, dextrose, fructose, xylitol, mannitol, sorbitol, calcium sulphate, starches, calcium carbonate, sodium carbonate, dextrates, dextrin, kaolin, lactitol, magnesium carbonate, magnesium oxide, maltitol, maltodextrin and maltose.

Suitable lubricants include magnesium stearate and sodium stearyl fumarate. Suitable glidants include colloidal silica and talc.

Suitable welting agents include sodium lauryl sulphate and docusate sodium. Suitable gas producers include sodium bicarbonate and citric acid.

The pharmaceutically active ingredient may be selected from a wide range of substances which may be administered orally. Suitable ingredients include acid-peptic and motility influencing agents, laxatives antidiarrhoeials, colorectal agents, pancreatic enzymes and bile acids, antiarrhythmics, antianginals, diuretics, anti-hypertensives, anti-coagulants, anti-thrombotics, fibrinolytics, haemostatics, hypolipidaemic agents, anti-anaemia and neurotropenia agents, hypnotics, anxiolytics, anti-psychotics, anti-depressants, anti-emetics, anti-convulsants, CNS stimulants, analgesics, anti-pyretics, anti-migraine agents, non-steroidal anti-inflammatory agents, anti-gout agents, muscle relaxants, neuro-muscular agents, steroids, hypoglycaemic agents, hyperglycaemic agents, diagnostic agents, antibiotics, anti-fungals, anti-malarials, anti-virals, immunosuppressants, nutritional agents, vitamins, electrolytes, anorectic agents, appetite suppressants, bronchodilators, expectorants, anti-tussives, mucolytic, decongestants, anti-glaucoma agents, oral contraceptive agents, diagnostic and neoplastic agents.

The electrostatic application of powder material to a substrate is known. Methods have already been developed in the fields of electrophotography and electrography and examples of suitable methods are described, for example, in Electrophotography and Development Physics, Revised Second Edition, by L. B. Schein, published by Laplacian Press, Morgan Hill Calif. The electrostatic application of powder material to a solid dosage form is known and techniques are disclosed, for example, in GB9929946.3, WO92/14451, WO96/35413, WO96/35516 and PCT/GB01/00425, and British Patent Application No. 9929946.3.

For example, WO92/14451 describes a process in which the cores of pharmaceutical tablets are conveyed on an earthed conveyor belt and electrostatically charged powder is deposited on the cores to form a powder coating on the surface of the cores.

A powder material for electrostatic application to a substrate should have certain properties. For example, the electrical properties of the powder material should be such as to make the powder material suitable for electrostatic application, and other properties of the powder material should be such that the material can be secured to the substrate once electrostatic application has taken place.

WO96/35413 describes a powder material which is especially suitable for electrostatic application to a poorly-conducting (non-metal) substrate such as a pharmaceutical tablet. Because it may be difficult to find a single component capable of providing the powder material with all the desired properties, the powder material comprises a number of different components which together are capable of providing the material with all or at least as many as possible of the desired properties, the components being co-processed to form “composite particles”. For example, the powder material may comprise composite particles including one component which is fusible to form a continuous film on the surface of the substrate, and another component which has desirable electrical properties.

A potential disadvantage of the above mentioned powder materials, however, is that they are not readily adaptable to changes in formulation. The formulation of a powder material may be changed for a number of different reasons. For example, if the material is a coloured material, there may be a change in the colourant, or if the material is an active material, for example a physiologically active material there may be a change in the type of active material, or in the concentration of that active material. Because all the components of the powder material are intimately mixed, any change in the components will alter the material's electrical properties and hence its performance in electrostatic application. Whenever there is a change in formulation, it may therefore be necessary, for optimum performance, to adjust the content of the component(s) that make the material suitable for electrostatic application, or perhaps even to use a different component.

PCT/GB01/00425 discloses a method of electrostatically applying a powder material to a substrate, wherein at least some of the particles of the material comprise a core and a shell surrounding the core, the core and the shell having different physical and/or chemical properties.

Where the particles of the powder material comprise a core and a shell surrounding the core, it is possible to place those components which are likely to be altered, for example colourant in the core, and to provide a more universal shell composition which is suitable for use with various core compositions, so that alterations may be made to the components that are in the core without substantially affecting the overall suitability of the powder material; thus, the shell ensures that the change in composition of the core does not affect the performance of the material in electrostatic application. Accordingly, alterations to one component of the powder material may be made with minimum alteration in the amounts of other components.

Generally, the powder material includes a component which is fusible, and that component may be present in the shell or in the core or in both the shell and the core. Advantageously, the fusible component is treatable to form a continuous film coating. Examples of suitable components are as follows: polyacrylates, for example polymethacrylates; polyesters; polyurethanes; polyamides, for example nylons; polyureas; polysulphones; polyethers; polystyrene; polyvinylpyrrolidone; biodegradable polymers, for example polycaprolactones, polyanhydrides, polylactides, polyglycolides, polyhydroxybutyrates and polyhydroxyvalerates; sugars, for example lactitol, sorbitol xylitol, galactitol, maltitol, fructose, xylose and galactose; hydrophobic waxes and oils, for example vegetable oils and hydrogenated vegetable oils (saturated and unsaturated fatty acids) e.g. hydrogenated castor oil, carnauba wax, and beeswax; hydrophilic waxes; polyalkenes and polyalkene oxides; polyethylene glycol. Clearly there may be other suitable materials, and the above are given merely as examples. One or more fusible materials may be present. Preferred fusible materials generally function as a binder for other components in the powder.

In general the powder material should contain at least 30%, usually at least 35%, advantageously at least 80%, by weight of material that is fusible, and, for example, fusible material may constitute up to 95%, e.g. up to 85%, by weight of the powder. Wax, if present, is usually present in an amount of no more than 6%, especially no more than 3% by weight, and especially in an amount of at least 1% by weight, for example 1 to 6%, especially to 1 to 3%, by weight of the powder material.

Of the materials mentioned above, polymer binders (also referred to as resins) should especially be mentioned. Examples include polyvinylpyrrolidone, hydroxypropyl cellulose, hydroxypropyl methylcellulose phthalate, hydroxypropyl methylcellulose acetate succinate and methacrylate polymers, for example an ammonio-methacrylate copolymer, for example those sold under the name Eudragit.

Often resin will be present with a wax as an optional further fusible component in the core; the presence of a wax may, for example, be useful where fusing is to take place by a contact system for example using a heated roller, or where it is desired to provide a glossy appearance in the fused film. The fusible component may comprise a polymer which is cured during the treatment, for example by irradiation with energy in the gamma, ultra violet or radio frequency bands. For example, the core may comprise thermosetting material which is liquid at room temperature and which is hardened after application to the substrate.

Preferably, the powder material includes a material having a charge-control function. That functionality may be incorporated into a polymer structure, as in the case of Eudragit resin mentioned above, and/or, for a faster rate of charging, may be provided by a separate charge-control additive. Material having a charge-control function may be present in the shell or in the core or in both shell and core. Examples of suitable charge-control agents are as follows: metal salicylates, for example zinc salicylate, magnesium salicylate and calcium salicylate; quaternary ammonium salts; benzalkonium chloride; benzethonium chloride; trimethyl tetradecyl ammonium bromide (cetrimide); and cyclodextrins and their adducts. One or more charge-control agents may be used. Charge-control agent may be present, for example, in an amount of up to 10% by weight, especially at least 1% by weight, for example from 1 to 2% by weight, based on the total weight of the powder material.

The powder material may also include a flow aid. The flow aid reduces the cohesive and/or other forces between the particles of the material to improve the flowability of the powder. Suitable flow aids (which are also known as “surface additives”) are, for example, as follows: colloidal silica; metal oxides, e.g. fumed titanium dioxide, zinc oxide or alumina; metal stearates, e.g. zinc, magnesium or calcium stearate; talc; functional and non-functional waxes, and polymer beads, e.g. poly-methyl methacrylate beads, fluoropolymer beads and the like. Such materials may also enhance tribocharging. A mixture of flow aids, for example silica and titanium dioxide, should especially be mentioned. The powder material may contain, for example, 0 to 3% by weight. advantageously at least 0.1%, e.g. 0.2 to 2.5%, of surface additive flow aid.

Often the powder material includes a colourant and/or an opacifier. When the powder comprises a core and shell such components are preferably present in the core. Examples of suitable colourants and opacifiers are as follows: metal oxides, e.g. titanium dioxide, iron oxides; aluminium lakes, for example, indigo carmine, sunset yellow and tartrazine; approved food dyes; natural pigments. A mixture of such materials may be used if desired. Opacifier preferably constitutes no more than 50%, especially no more than 40%, more especially no more than 30%, for example no more than 10% by weight of the powder material, and may be used, for example, in an amount of at least 5% by weight of the powder. Titanium dioxide is an especially useful opacifier, providing white colour and having good hiding power and tinctorial strength. Colourant present with opacifier may, for example, constitute no more than 10%, preferably from 1 to 5%, by weight of the powder. If there is no opacifier, the colourant may be, for example, 1 to 15%, e.g. 2 to 15%, especially 2 to 10%, by weight of the powder. To achieve optimum colour, amounts of up to 40% by weight of colourant may be needed in some cases, for example if inorganic pigments, e.g. iron oxides, are used. However, the powder material usually contains, for example, from 0 to 25% by weight in total of colourant and/or opacifier.

The powder material may also include a dispersing agent, for example a lecithin. The dispersing agent is preferably present with the colourant/opacifier (that is, preferably in the core), serving to improve the dispersion of the colourant and opacifier, more especially when titanium dioxide is used. The dispersing component is preferably a surfactant which may be anionic, cationic or non-ionic, but may be another compound which would not usually be referred to as a “surfactant” but has a similar effect. The dispersing component may be a co-solvent. The dispersing component may be one or more of, for example, sodium lauryl sulphate, docusate sodium, Tweens (sorbitan fatty acid esters), polyoxamers and cetostearyl alcohol. Preferably, the powder material includes at least 0.5%, e.g. at least 1%, for example from 2% to 5%, by weight of dispersing component, based on the weight of the powder material. Most often it is about 10% by weight of the colourant and opacifier content.

The powder material may also include a plasticiser, if necessary, to provide appropriate rheological properties. A plasticiser may be present in the core and/or the shell, but usually, if present, a plasticiser is included with resin used for the core to provide appropriate rheological properties, for example for preparation of the core by extrusion in a melt extruder. Examples of suitable plasticisers include polyethylene glycols, triethyl citrate, acetyltributyl citrate, acetyltriethyl citrate, tributyl citrate, diethyl phthalate, dibutyl phthalate, dimethyl phthalate, dibutyl sebacate and glyceryl monostearate.

A plasticiser may be used with a resin in an amount, for example, of up to 50% by weight of the total of that resin and plasticiser, the amount depending inter alia on the particular plasticisers used. The powder may contain an amount of up to 50% by weight of plasticiser.

The powder coating material may further include one or more taste modifiers, for example aspartame, acesulfame K, cyclamates, saccharin, sugars and sugar alcohols or flavourings. Preferably there is no more than 5%, more preferably no more than 1 %, of flavouring based on the weight of the powder material, but larger or smaller amounts may be appropriate, depending on the particular taste modifier used.

If desired the powder material may further include a filler or diluent. Suitable fillers and diluents are essentially inert and low cost materials with generally little effect on the colour or other properties of the powder. Examples are as follows: alginic acid; bentonite; calcium carbonate; kaolin; talc; magnesium aluminium silicate; and magnesium carbonate.

The particle size of the powder material has an important effect on the behaviour of the material in electrostatic application. Although materials having a small particle size are recognised as having disadvantages such as being more difficult to produce and to handle by virtue of the material's cohesiveness, such material has special benefits for electrostatic application and the benefits may more than counter the disadvantages. For example, the high surface to mass ratio provided by a small particle increase the electrostatic forces on the particle in comparison to the inertial forces. Increasing the force on a particle has the benefit of increasing the force that causes it to move into contact with the substrate, whilst a reduction in the inertia reduces the force needed to accelerate a particle and reduces the likelihood of a particle arriving at the substrate bouncing back off the substrate. However, very small particle sizes may not be achievable where the coating material comprises a high proportion of a particular ingredient, for example a high proportion of active material.

Preferably, at least 50% by volume of the particles of the material have a particle size no more than 100 μm. Advantageously, at least 50% by volume of the particles of the material have a particle size in the range of 5 μm to 40 μm. More advantageously, at least 50% by volume of the particles of the material have a particle size in the range of 10 to 25 μm.

Powder having a narrow range of particle size should especially be mentioned. Particle size distribution may be quoted, for example, in terms of the Geometric Standard Deviation (“GSD”) ratios d₉₀/d₅₀ or d₅₀/d₁₀ where d₉₀ denotes the particle size at which 90% by volume of the particles are below this figure (and 10% are above), d₁₀ represents the particle size at which 10% by volume of the particles are below this figure (and 90% are above), and d₅₀ represents the mean particle size. Advantageously, the mean (d₅₀) is in the range of from 5 to 40 μm, for example, from 10 to 25 μm. Preferably, d₉₀/d₅₀ is no more than 1.5, especially no more than 1.35, more especially no more than 1.32, for example in the range of from 1.2 to 1.5, especially 1.25 to 1.35, more especially 1.27 to 1.32, the particle sizes being measured, for example, by Coulter Counter. Thus, for example, the powder may have d₅₀=10 μm, d₉₀=13 μm, d₁₀=7 μm, so that d₉₀/d₅₀=1.3 and d₅₀/d₁₀=1.4.

The powder material is fusible so that it is treatable to form a continuous film coating.

It is important that the powder can be fused or treated without degradation of any active material in the powder and without degradation of the tablet core. For some materials it may be possible for the treatment step to involve temperatures up to and above 250° C. Preferably, however, the powder material is fusible at a pressure of less than 100 lb/sq. inch, preferably at atmospheric pressure, at a temperature of less than 200° C., and most commonly below 150° C., and often at least 80° C., for example in the range of from 100 to 140° C.

Fusing of the powder material may be carried out by any of a number of different fusing methods. If desired, rupture of the shell and fusing of the material may be carried out in a single step. The powder material is preferably fused by changing the temperature of the powder, for example by radiant fusing using electromagnetic radiation, for example infra red radiation or ultra-violet radiation, or conduction or induction, or by flash fusing. The amount of heat required may be reduced by applying pressure to the powder material, for example by cold pressure fusing or hot roll fusing.

Preferably, the powder material has a glass transition temperature (Tg) in the range of 40° C. to 120° C. Advantageously, the material has a Tg in the range of 50° C. to 100° C. A preferred minimum Tg is 55° C., and a preferred maximum Tg is 70° C. Accordingly, more advantageously, the material has a Tg in the range of 55° C. to 70° C. Generally, the powder material should be heated to a temperature above its softening point, and then allowed to cool to a temperature below its Tg.

The powder material once fused is substantially insoluble, preferably completely insoluble in aqueous media at temperatures up to the body temperature. Thus, the powder material will comprise a significant amount of an insoluble material. Preferred material comprises a polymer resin selected from polymethacrylates, polyvinyl alcohols and esters, cellulose and its derivatives, cellulose ethers and esters and cellulose acetate phthalate.

The electrostatic coating of the tablet core by the powder material may be conducted by any of the methods disclosed in the above referenced patents. The partial coating of the tablet core may be achieved by the use of a mask. However, preferably the partial coating is achieved by coating one face and the sides of a tablet core in accordance with the first stage of coating as described in the above mentioned patents. Thereafter the electrostatically deposited powder is fused to form a tablet core having a casing covering one face and the sides, leaving the other face exposed.

The invention will be illustrated by the following Examples and drawings in which:

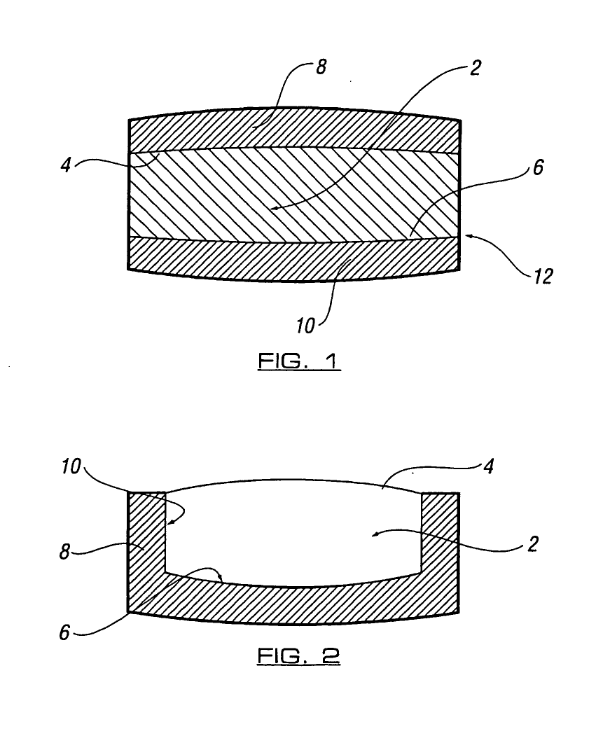

FIGS. 1 and 2 represent diagrams of different solid dosage forms in accordance with the invention.

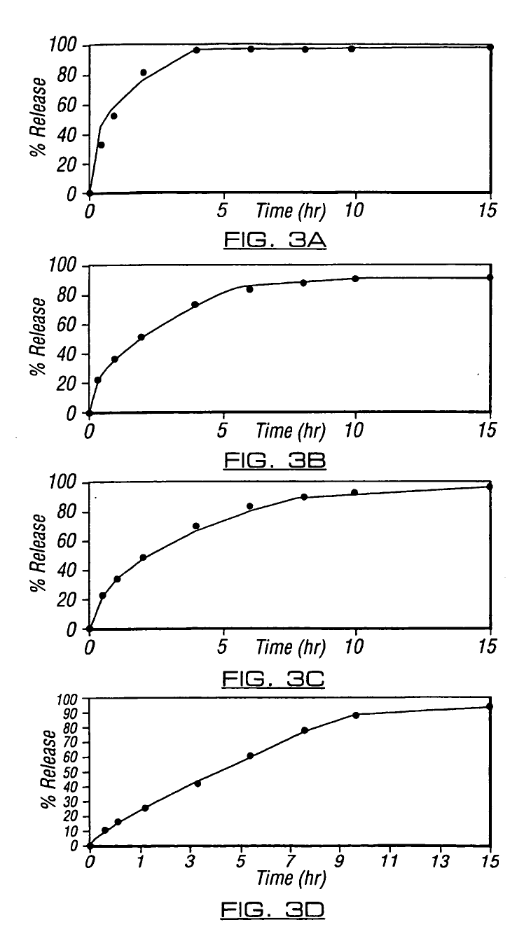

FIG. 3a shows the release profile of a tablet core containing diltiazem and mixed hydrophobic/hydrophilic polymers as described in Example 1.

FIG. 3b to 3g shows the release profile of a coated tablet containing diltiazem and mixed hydrophobic/hydrophilic polymers on the two major faces of the tablets, with 0.5%, 0.7%, 1.4%, 1.9%, 2.3% and 2.8% weight gains respectively as described in Example 1.

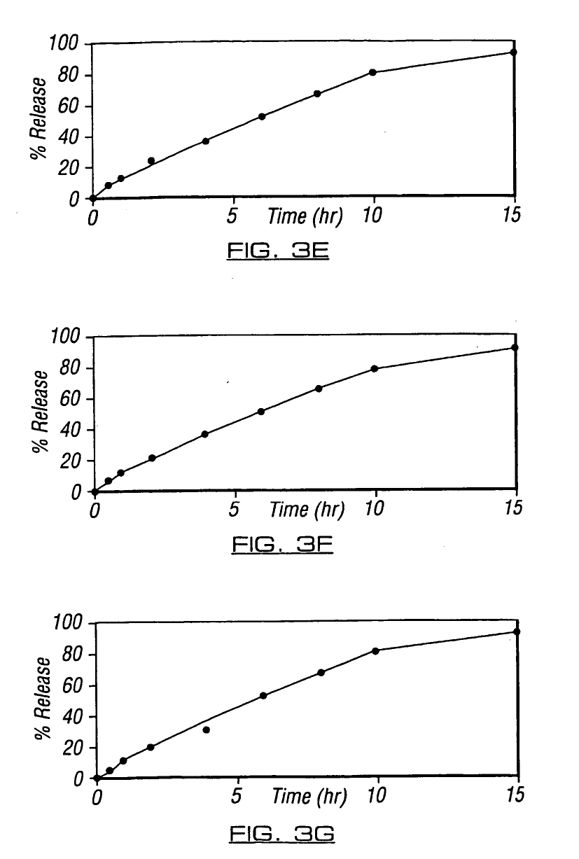

FIGS. 4a and 4b show the release profiles of a tablet core and the coated tablet containing salbutamol and hydrophobic matrix as described in Example 2.

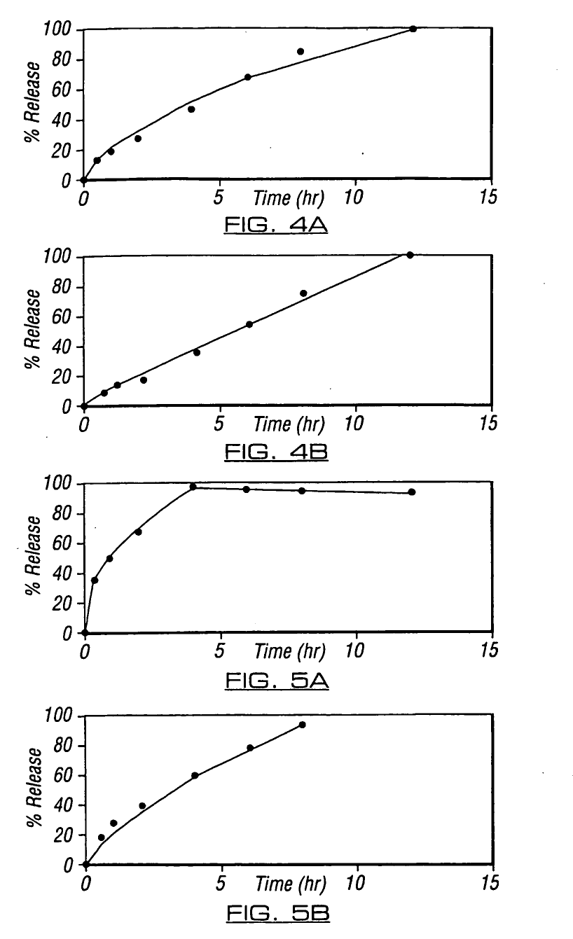

FIGS. 5a and 5b show the release profiles of a hydrophilic tablet core and the coated tablet as described in Example 3.

FIGS. 6a and 6b show the release profiles of a mixed hydrophilic/hydrophobic tablet core and the coated tablet as described in Example 4.

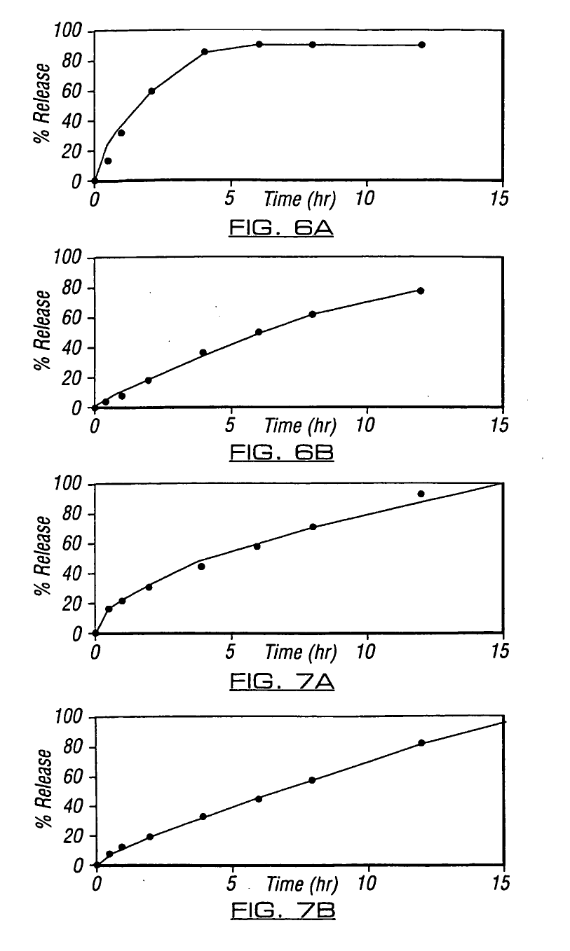

FIGS. 7a and 7b show the release profiles of a hydrophilic tablet core and the coated tablet as described in Example 5.

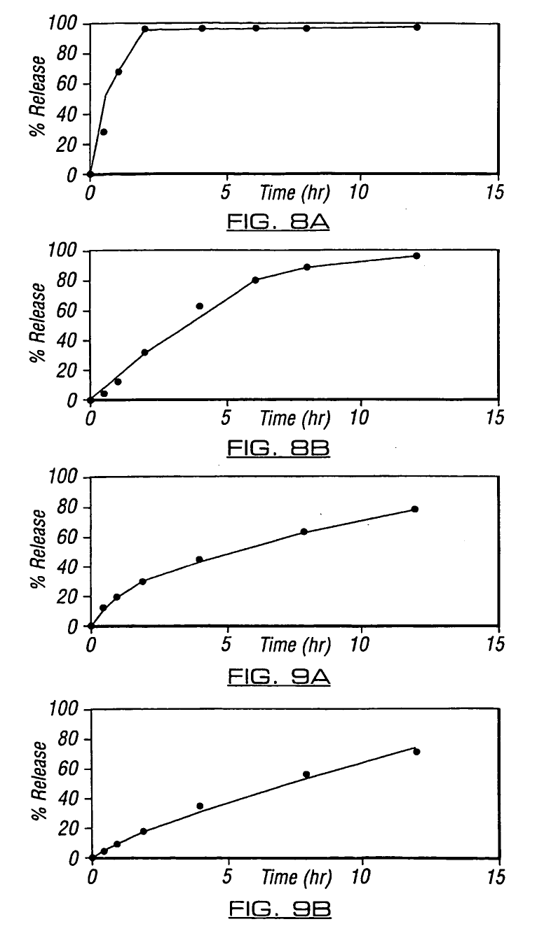

FIGS. 8a and 8b show the release profiles of a hydrophobic tablet core and the coated tablet as described in Example 6.

FIGS. 9a and 9b show the release profiles of a hydrophilic tablet core and the coated tablet as described in Example 7.

FIG. 1 shows a dosage form in accordance with the invention comprises a tablet core (2), which is circular in shape and has opposing major faces (4, 6), which are convex. The faces (4, 6) are coated with an insoluble coating (8, 10) leaving the sidewall (12) exposed.

Referring to FIG. 2, which illustrates a cross-section through a dosage form with the invention, a tablet core (2) has a circular cross-section and opposing major convex surfaces (4, 6). The insoluble casing (8) extends over the major surface (6) and side (10) leaving major surface (4) exposed.

The following materials were used in the Examples:

| | |
|---|---|
| Eudragit RS30D | a methacrylate polymer commercially available from |
| | Rhom |
| Methocel 66LV | hydroxy propyl methyl cellulose commercially |
| | available from Dow Chemicals |
| Methocel K4M | hydroxy propyl methyl cellulose commercially |
| | available from Dow Chemicals |
| Methocel K15M | hydroxy propyl methyl cellulose commercially |
| | available from Dow Chemicals |
| Methocel K100M | hydroxy propyl methyl cellulose commercially |
| | available from Dow Chemicals |
| Eudragit RSPO | a methacrylate copolymer commercially available from |
| | Rohm |
| Eudragit RLPO | a methacrylate copolymer commercially available from |
| | Rohm |
| Eudragit NE30D | a methacrylate copolymer commercially available from |
| | Rohm |

In the Examples all-parts and percentages are by weight unless otherwise stated.

### EXAMPLE 1

### Effect of Coating Thickness: Mixed Polymer Coated on Both Major Faces with Insoluble Coat

The construction of the dosage form is shown in FIG. 1.

Tablet cores were formulated by mixing:

| | |
|---|---|
| Diltiazem HCl | 17.14% |
| Eudragit RS30D | 5.00% (added as 30% aqueous |
| | solution) |
| Methocel 66LV | 2.00% |
| Microcrystalline cellulose | 20.00% |
| DCPA (dihydrogen | 44.86% |
| calcium phosphate anhydrous) | |
| Eudragit RSPO | 10.00% |

The mixture was oven dried to approximately 1% moisture. 1.00% magnesium stearate was added to the dried granules and mixture was compressed into 10 mm standard biconvex tablets. The tablet cores had an average weight of about 350 mg and a hardness of about 19 kp.

Two coating formulations for the casing were prepared. Coating Formulation I had the following composition:

| | |
|---|---|
| Eudragit RSPO | 89.8% |
| Polyethylene glycol 6000 | 2.7% |
| Titanium dioxide | 5.0% |
| Indigo Carmine (blue) | 2.5% |

Coating Formulation II was blended with 0.2% Aerosil 200 before application and it has the following composition:

| | |
|---|---|
| Eudragit RSPO | 87.2% |
| Triethyl citrate | 5.37% |
| Titanium dioxide | 5.0% |
| Sunset yellow (orange) | 2.5% |

To prepare the coating powder, the above ingredients were weighed, blended, then extruded. The extrudates were pin-milled, micronised and classified in an air jet mill to give a median particle size of approximately 10 μm.

A blend containing 4.5% of Coating Formulation I and 95.5% of a silicone coated ferrite was prepared. The tablets were coated electrostatically using the coat/carrier blend in a conventional dual component delivery device adapted from the electrophotographic industry such that the Coating Formulation I (without ferrite carrier) was applied to both faces of the tablet leaving the sides uncoated. Details of the coating process are disclosed in British Patent Application No. 9929946.3. The coat was fused onto the tablets at approximately 100° C., to provide a range of coating thickness between 10 and 60 μm. The tablets were then turned over and the second coating applied on the other sides of the tablets by the same technique using Coating Forrnulation II.

Six uncoated and six coated tablets of each coating thickness (as expressed by % weight gain) were assessed for release rate in 900 ml of demineralised water at 37° C. using USP Apparatus II (paddles) at 50 rpm as described above and diltiazem analysed by UV.

The results are summarised in Table 1.

TABLE 1

| | | Coat thickness I | | Coat thickness II | | |
|---|---|---|---|---|---|---|
| Sample | | (centre μm) | | (edges μm) | | Release |
| % Weight gain | | Side 1 | Side 2 | Side 1 | Side 2 | exponent (n) |
| a | 0 (core) | 0 | 0 | 0 | 0 | 0.37 |
| b | 0.5% | 10 | 14 | 12 | 13 | 0.50 |
| c | 0.7% | 43 | <10 | 14 | <10 | 0.45 |
| d | 1.4% | 33 | 33 | 23 | 30 | 0.78 |
| e | 1.9% | 26 | 28 | 22 | 26 | 0.80 |
| f | 2.3% | 33 | 30 | 26 | 23 | 0.79 |
| g | 2.8% | 39 | 64 | 30 | 44 | 0.82 |

The release rate with time for each different tablet is shown in FIGS. 3a to 3g.

These results demonstrate that electrostatic application of a thin coat on the selected surface of a tablet core, which had a release profile close to first order release, i.e. release exponent n=0.30-0.65, resulted in a dosage form having a release profile substantially close to zero order, i.e. n=0.7-1.0.

It is known that conventional solvent coating results in a substantial thick coating with a weight gain of at least 5%, or frequently above 10% for modified release systems. The present results demonstrate that zero order release can be achieved with a very thin coat by electrostatic coating and the release profiles are insensitive to coating thickness provided a continuous and complete coverage of the coated area is achieved, i.e. the deposition of several layer coating powder to give a fused coat of approximately 20 μm thick.

### Example 2

### Hydrophobic Tablet Core Coated on Both Major Faces with Insoluble Coat

The construction of the dosage form is as illustrated in FIG. 1.

Tablet cores were formulated by mixing:

2.74% Salbutamol sulphate

71.26% anhydrous DCPA

25% Eudragit RLPO

1% magnesium stearate

The mixture was compressed into 10 mm standard biconvex tablets. The tablet cores had an average weight of about 350 mg and a hardness of 10 kp. Coating Formulation III was used to coat both sides of the tablets as described in Example 1 to provide a casing of 20 to 50 μm. Coating

Formulation III comprises:

84.0% Eudragit RSPO

8.5% polyethylene glycol 6000

5.0% titanium dioxide

2.5% sunset yellow lake

Three uncoated and three coated tablets were assessed for release rate in 500 ml of demineralised water at 37° C. using USP Apparatus II (paddles) at 50 rpm and the release rate analysed by UV over 12 hours. The release rate with time is shown in FIGS. 4a and 4b. The following kinetic models can be used to describe the release characteristics from the cores and coated tablets (0-100% release range):

Core: y=22.3t^{0.59}

Coated: y=10.8t^{0.90}

It is evident that electrostatic application of a thin coat substantially modified the release profile of a tablet core comprising hydrophobic polymers.

### Example 3

### Hydrophobic Tablet Core Comprising a Different Active Coated on Both Major Faces with Insoluble Coat

The construction of the dosage form is as shown in FIG. 1.

Tablet cores were formulated by mixing:

17.14% ditiazem hydrochloride

20.00% microcrystalline cellulose

51.86% anhydrous DCPA

10.00% Eudragit RS30D added as 30% aqueous dispersion

The mixture was oven dried to approximately 1% moisture. 1% magnesium stearate was added to the dried granules and the mixture compressed into 10 mm standard biconvex tablets. The tablet cores had an average weight of about 350 mg and a hardness of 19 kp.

A blend containing 10% of Coat Formulation III as described in Example 2 and 90% of a silicone coated strontium ferrite carrier was prepared. The tablet cores were coated on both major faces using the materials and method as described in Example 2 with the exception that the coat was fused at 120° C. The coating thickness was in the range 20 to 50 μm

Six cores and six coated tablets were assessed for release rate in 900 ml of demineralised water at 37° C. using USP Apparatus II (paddles) at 50 rpm and the release rate analysed by HPLC.

The release of diltiazem over time is shown in FIGS. 5a and 5b respectively. The following kinetic models can be used to describe the release characteristics from the cores and coated tablets (0-90% release range):

Core: y=52t^{0.43}

Coated: y=22t^{0.70}

It is evident that electrostatic application of a thin coat on the major faces of the tablet substantially modified the release profile of a tablet core comprising hydrophobic polymers.

### Example 4

### Mixed Hydrophobic/Hydrophilic Tablet Core Coated on Both Major Faces with Insoluble Coat

The construction of the dosage form is as illustrated in FIG. 1.

The tablet cores were formulated by mixing:

17.14% Diltiazem hydrochloride

20.00% microcrystalline cellulose

50.86% anhydrous DCPA

1% Methocell K15M

10% (as solid) Eudragit NE30D (30% aqueous dispersion)

The mixture was oven dried to approximately 1% moisture. 1% magnesium stearate was added to the dried granules and the mixture was compressed into 10 mm standard biconvex tablets. The tablet cores had an average weight of about 350 mg and a hardness of about 19 kp.

The tablet cores were coated using the materials and method as described in Example 3. The release rate was determined as described in Example 3 and the results are shown in FIGS. 6a and 6b. The following kinetic models can be used to describe the release characteristics from the cores and coated

tablets (0-80% release):

Core: y=38.5t^{0.56}

Coated: y=10.5t^{0.85}

It is evident that electrostatic application of a thin coat on the major faces of tablets substantially modified the release profile of a tablet core comprising mixed hydrophilic/hydrophobic polymers.

### Example 5

### Hydrophilic Tablet Core Coated on Both Faces of the Tablet

The construction of the dosage form is shown in FIG. 1.

Tablet cores were formulated by mixing:

2.74% Salbutamol sulphate

46.26% anhydrous lactose DC (directly compressible)

50.00% Methocel K4M

1.00% magnesium stearate

The mixture was compressed into 10 mm standard biconvex tablets. The tablet cores had an average weight of 350 mg and a hardness of about 19 kp. Coating Formulation III was applied on the major opposite faces of the tablet core as described in Example 2 to provide a coating having a thickness in the range of from 20 to 50 μm.

The release rate with time was determined according to the method described in Example 2 and the results are shown in FIGS. 7a and 7b respectively. The following kinetic models can be used to describe the release characteristics from the cores and coated tablets (0-80%):

Core: y=22.1t^{0.56}

Coated: y=11.0t^{0.80}

It is evident that electrostatic application of a thin coat on the major faces of tablets substantially modified the release profile of a tablet core comprising hydrophilic polymers.

### Example 6

### Hydrophobic Tablet Core Coated on One Face and the Sides of the Tablet

The construction of the dosage form is shown in FIG. 2.

Tablet cores were formulated by mixing:

2.74% Salbutamol sulphate

71.26% anhydrous DCPA

25.00% Eudragit RLPO

1.00% magnesium stearate

The mixture was compressed into 10 mm standard biconvex tablets. The tablet cores had an average weight of 350 mg and a hardness of about 11 kp. Coating Formulation III was applied on one major face and the sides of the tablet core and the method of coating was as described in Example 2 to provide a coating thickness in the range 20 to 50 μm.

The release rate with time was determined according to the method described in Example 2 and the results are shown in FIGS. 8a and 8b respectively.

The following kinetic models can be used to describe the release characteristics from the cores and coated tablets (0-95% for the core and 0 -80% for the coated tablet):

Core: y=70t^{0.47}

Coated: y=16.3t^{0.90}

It is evident that electrostatic application of a thin coat on the major faces of tablets substantially modified the release profile of a tablet core comprising hydrophilic polymers.

### Example 7

### Hydrophilic Tablet Core Coated on One Face and the Sides of the Tablet

The construction of the dosage form is shown in FIG. 2.

Tablet cores were formulated by mixing:

2.74% Salbutamol sulphate

46.26% anhydrous lactose DC (directly compressible)

50.00% Methocel K100M

1.00% magnesium stearate

The mixture was compressed into 10 mm standard biconvex tablets. The tablet cores had an average weight of 350 mg and a hardness of about 15 kp. Coating formulation III was applied on one face and the sides of the tablet core and the method of coating was as described in Example 2 to provide a coating thickness in the range 20 to 50 μm.

The release rate with time was determined according to the method described in Example 2 and the results are shown in FIGS. 9a and 9b respectively. The following kinetic models can be used to describe the release characteristics from the cores and coated tablets (0-70%):

Core: y=21.0t^{0.55}

Coated: y=10.9t^{0.78}

It is evident that electrostatic application of a thin coat on one face and the sides of tablets substantially modified the release profile of a tablet core comprising hydrophilic polymers.

1. A controlled release dosage form comprising:
(i) a tablet core comprising a pharmaceutically active ingredient and one or more pharmaceutically acceptable matrix forming polymers, the tablet core releases the active ingredient with a release profile of active ingredient for 0 to at least 50% by weight release of active ingredient defined by the equations
y=k*tⁿ
in which y is the fraction of active ingredient released
k is the kinetic constant
t is time
n is the release exponent
and n is the range 0.30 to 0.65
(ii) a substantially insoluble casing extended over the tablet core covering between 25 to 99% of the surface area of the tablet core, the casing resulting from electrostatic deposition of a powder comprising fusible particles onto the tablet core and fusing the particles to form a thin film such that the said electrostatic coated tablet releases the active ingredient with a release profile of active ingredient for 0 to at least 50% by weight release of active ingredient defined by the equations
y=k*tⁿ
in which y is the fraction of active ingredient released
k is the kinetic constant
t is time
n is the release exponent
and n is the range 0.70 to 1.0.

2. A solid pharmaceutical dosage form as claimed in claim 1 in which the insoluble casing covers from 65 to 95% of the surface area of the tablet core.

3. A solid pharmaceutical dosage form as claimed in claim 1 in which the tablet core comprises two major opposing surfaces separated by one or more sidewalls at least the major surfaces being covered by the casing.

4. A solid pharmaceutical dosage form as claimed in claim 1 in which the tablet core comprises two major opposing surfaces separated by one or more sidewalls one major surface and the one or more sidewalls being covered by the casing.

5. A solid pharmaceutical dosage form as claimed in claim 1 in which the controlled release dosage form has a release profile in which n=0.7 to 1.0 over from 0 to at least 70% by weight release of the active ingredient.

6. A solid pharmaceutical dosage form as claimed in claim 1 in which the release profile of the controlled release dosage form requires at least 4 hours to achieve 70% by weight release of active ingredient.

7. A solid pharmaceutical dosage form as claimed in claim 1 in which the tablet core comprises a binder selected from acacia, alginic acid, carboxymethylcellulose, hydroxyethylcellulose, hydroxypropylcellulose, dextrin, ethylcellulose, gelatin, glucose, guar gum, hydrogenated vegetable oil, hydroxypropylmethylcellulose, magnesium aluminium silicate, Maltodextrin, methylcellulose, polyethylene oxide, povidone, sodium alginate and hydrogenated vegetable oils.

8. A solid pharmaceutical dosage form as claimed in claim 1 in which the tablet core additionally comprises a release rate controlling polymer is selected from polymethacrylates, ethylcellulose, hydroxypropylmethylcellulose, methylcellulose, hydroxyethylcellulose, hydroxypropylcellulose, sodium carboxymethylcellulose, calcium carboxymethylcellulose, acrylic acid polymer, polyethylene glycol, polyethylene oxide, carrageenan, cellulose acetate, glyceryl monostearate and zein.

9. A solid pharmaceutical dosage form as claimed in claim 1 in which the tablet core additionally comprises a diluent selected from lactose, cellulose, dicalcium phosphate, sucrose, dextrose, fructose, xylitol, mannitol, sorbitol, calcium sulphate, starches, calcium carbonate, sodium carbonate, dextrates, dextrin, kaolin, lactitol, magnesium carbonate, magnesium oxide, maltitol, maltodextrin and maltose.

10. A solid pharmaceutical dosage form as claimed in claim 1 in which the tablet core comprises a hydrophobic matrix containing an active ingredient, a hydrophilic matrix containing an active ingredient, or a mixture of hydrophilic and hydrophobic materials.

11. A solid pharmaceutical dosage form as claimed in claim 1 in which the active ingredient is selected from acid-peptic and motility influencing agents, laxatives, antidiarrheials, colorectal agents, pancreatic enzymes and bile acids, antiarrhythmics, antianginals, diuretics, anti-hypertensives, anti-coagulants, anti-thrombotics, fibrinolytics, haemostatics, hypolipidaemic agents, anti-anaemia and neurotropenia agents, hypnotics, anxiolytics, anti-psychotics, anti-depressants, anti-emetics, anti-convulsants, CNS stimulants, analgesics, anti-pyretics, anti-migraine agents, non-steroidal anti-inflammatory agents, anti-gout agents, muscle relaxants, neuro-muscular agents, steroids, hypoglycaemic agents, hyperglycaemix agents, diagnostic agents, antibiotics, anti-fungals, anti-malarials, anti-virals, immunosuppressants, nutritional agents, vitamins, electrolytes, anorectic agents, appetite suppressants, bronchodilators, expectorants, anti-tussives, mucolytes, decongestants, anti-glaucoma agents, oral contraceptive agents, diagnostic and neoplastic agents.

12. A solid pharmaceutical dosage form as claimed in claim 1 in which the tablet core comprises a polymeric material which swells on contact with aqueous liquid, said swellable polymeric material being selected from cross-linked sodium carboxymethylcellulose, cross-linked hydroxypropylcellulose, high molecular weight hydroxypropylcellulose, carboxymethylamide, potassium methacrylatedivinylbenzene copolymer, polymethylmethacrylate, cross-linked polyvinylpyrrolidone and high molecular weight polyvinylalcohols.

13. A solid pharmaceutical dosage form as claimed in claim 1 in which the casing comprises a polymer resin selected from polymethacrylates, cellulose and its derivatives, cellulose ethers and esters and cellulose acetate phthalate.

14. A solid pharmaceutical dosage form as claimed in claim 1 in which the casing additionally comprises one or more adjuvants selected from opacifiers, colourants, plasticisers, flow aids and charge control materials.

15. A solid pharmaceutical dosage form as claimed in claim 14 in which the casing comprises a plasticiser selected from polyethylene glycols, triethyl citrate, acetyltributyl citrate, acetyltriethyl citrate, tributyl citrate, diethyl phthalate, dibutyl phthalate, dimethyl phthalate, dibutyl sebacate and glyceryl monostearate.

16. A solid pharmaceutical dosage form as claimed in claim 1 in which the casing has an average thickness of from 20 to 50 μM.

17. A solid pharmaceutical dosage form as claimed in claim 1 in which the casing results in a weight gain of less than 4% by weight of the tablet core.
